(19) United States
(12) Patent Application Publication
(10) Pub. No.: US 2022/0145179 A1
(43) Pub. Date: May 12, 2022

(54) SYNTHESIS OF ARYL 1-(METHOXYMETHYL) VINYL KETONES AND THEIR USE AS INHIBITORS OF MILD STEEL CORROSION

(71) Applicants: SAUDI ARABIAN OIL COMPANY, Dhahran (SA); KING FAHD UNIVERSITY OF PETROLEUM & MINERALS, Dhahran (SA)

(72) Inventors: Mohammad Abu Jafar Mazumder, Hamilton (CA); Shaikh Asrof Ali, West Bengal (IN); Nurudeen Odewunmi, Al-Khobar (SA); Bader Alharbi, Dammam (SA); Norah Aljeaban, Al Khobar (SA); Tao Chen, Dhahran (SA); Salem Balharth, Al Khobar (SA)

(73) Assignees: SAUDI ARABIAN OIL COMPANY, Dhahran (SA); KING FAHD UNIVERSITY OF PETROLEUM & MINERALS, Dhahran (SA)

(21) Appl. No.: 17/096,073

(22) Filed: Nov. 12, 2020

(51) Int. Cl. C09K 15/08; C07C 45/66; C07C 45/65; C23F 11/04

(52) U.S. Cl. CPC C09K 15/08; C23F 11/04; C07C 45/65; C07C 45/66

(57) ABSTRACT

Methods for preparing alkenylphenone corrosion inhibiting compositions may include providing an arylacetone and reacting the arylacetone with formaldehyde in the presence of a strong base catalyst. Corrosion inhibiting compositions may include an alkenylphenone, which may be prepared by a method including providing an arylacetone and reacting the arylacetone with formaldehyde in the presence of a strong base catalyst. Methods of inhibiting corrosion of a steel surface of an oilfield equipment component may include contacting the steel surface with an aqueous solution comprising a corrosion inhibitor. The corrosion inhibitor may include a composition containing an alkenylphenone prepared by reacting an arylacetone with formaldehyde in the presence of a strong base catalyst.

### BACKGROUND

Mild steel is an inexpensive and commonly used steel alloy that is weldable, very hard and durable. However, mild steel generally exhibits poor corrosion resistance especially when mild steel is exposed to aqueous acidic liquids. As such, mild steel requires protection from corrosion when it is exposed to acidic materials. In particular, oil and gas exploration and production operations commonly use mild steel equipment. These operations also commonly require the treatment of formations with well fluids containing acids to stimulate oil and gas production. These well fluids are therefore corrosive media that attack the mild steel surfaces with which they come into contact as they create an environment in which the mild steel surfaces are more susceptible to corrosion.

In particular, corrosive well fluids may perforate or severely damage well equipment and thus reduce the efficiency of the corresponding operations. In addition, where the well fluids cause corrosion of well equipment having mild steel surfaces, the life of such equipment may be appreciably reduced. Accordingly, the protection of mild steel equipment against corrosion with effective inhibitors is highly desirable.

### SUMMARY

In one aspect, embodiments disclosed herein are directed to methods for preparing an alkenylphenone corrosion inhibiting composition. The methods may include providing an arylacetone having a structure represented by Formula (I):

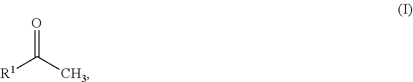

wherein R¹ is a substituted or unsubstituted aryl group having 6 to about 20 carbons. The methods may include reacting the arylacetone with formaldehyde in the presence of a strong base catalyst to form an alkenylphenone composition.

In another aspect, embodiments disclosed herein are directed to methods including forming an intermediate having a structure represented by Formula (II):

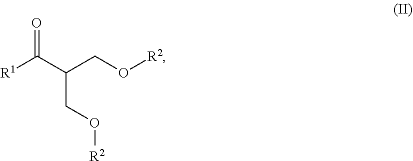

wherein R² is hydrogen or a substituted or unsubstituted alkyl group.

In another aspect, embodiments disclosed herein are directed to methods including forming an intermediate having a structure represented by Formula (III):

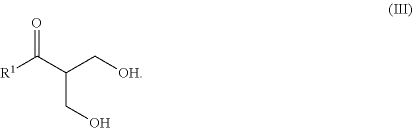

In another aspect, embodiments disclosed herein are directed to compositions including an alkenylphenone having a structure represented by Formula (IV):

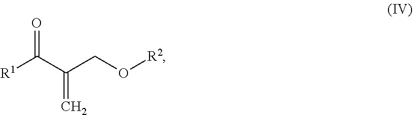

wherein R¹ is a substituted or unsubstituted aryl group having 6 to about 20 carbons, and R² is a substituted or unsubstituted alkyl group. In the compositions, the alkenylphenone may be prepared by a method including providing an arylacetone having a structure represented by Formula (I) and reacting the arylacetone with formaldehyde in the presence of a strong base catalyst to form an alkenylphenone composition.

In another aspect, embodiments disclosed herein are directed to methods of inhibiting corrosion of a steel surface of an oilfield equipment component. The methods may include contacting the steel surface with an aqueous solution comprising a corrosion inhibitor. The corrosion inhibitor may include a composition containing an alkenylphenone having a structure of Formula (IV) that is prepared by reacting an arylacetone of formula (I) with formaldehyde in the presence of a strong base catalyst.

Other aspects and advantages of this disclosure will be apparent from the following description made with reference to the appended claims.

### DETAILED DESCRIPTION

Embodiments in accordance with the present disclosure generally relate to alkenylphenone corrosion inhibiting compositions, their methods of preparation, and related methods of inhibiting corrosion.

In particular, the steel surface of certain oilfield equipment components, in particular mild steel equipment, may become perforated or severely damaged by corrosive well fluids, thus appreciably reducing their lifespan. Accordingly, the protection of mild steel equipment against corrosion with effective inhibitors is highly desirable.

Hence, there is a need for compositions and methods that may provide adequate inhibition for the corrosion of mild steel when in presence of corrosive well fluids. The present disclosure relates to methods for preparing an alkenylphenone corrosion inhibiting compositions. The methods may comprise providing an arylacetone having a structure represented by Formula (I):

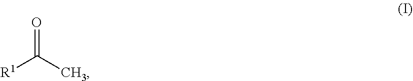

wherein R¹ is a substituted or unsubstituted aryl group having 6 to about 20 carbons; and
reacting the arylacetone with formaldehyde in the presence of a strong base catalyst to form an alkenylphenone composition.

The reaction to prepare the alkenylphenone of the corrosion inhibiting compositions may include forming intermediates having structures represented by Formulas (II) and (III):

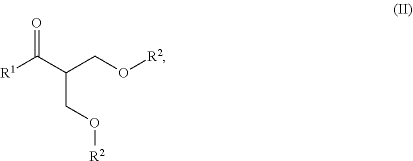

wherein R² is hydrogen or a substituted or unsubstituted alkyl group, or

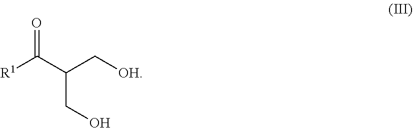

The present disclosure also relates to compositions including an alkenylphenone having a structure represented by Formula (IV):

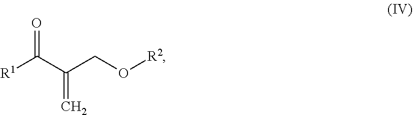

wherein R¹ is a substituted or unsubstituted aryl group having 6 to about 20 carbons, and R² is a substituted or unsubstituted alkyl group. The alkenylphenone of the compositions may be prepared by a method including providing an arylacetone having a structure represented by Formula (I) and reacting the arylacetone with formaldehyde in the presence of a strong base catalyst to form an alkenylphenone composition.

Arylacetone

In one or more embodiments of the present disclosure, the general structure of the arylacetone compounds is represented by Formula (I):

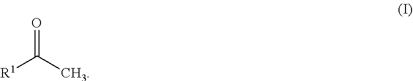

In some embodiments, R¹ may be a substituted or unsubstituted aryl group having 6 to about 20 carbons. In some embodiments, R¹ may be a substituted aryl group, wherein the aryl group is substituted by one or more of an alkyl group, an alkenyl group, an alkoxide group, a halogen group, an halogenoalkyl, and an aryl group. In some embodiments, R¹ may be a substituted or unsubstituted phenyl group, a substituted or unsubstituted biphenyl group, a substituted or unsubstituted naphthyl group, a substituted or unsubstituted triphenyl group, a substituted or unsubstituted terphenyl group, a substituted or unsubstituted anthracenyl group, a substituted or unsubstituted anthracenyl group, a substituted or unsubstituted indanyl group, or a substituted or unsubstituted indenyl group. In some embodiments, R¹ may be a phenyl group, a methoxyphenyl group, a tolyl group, a xylyl group, an ethylphenyl group, an isopropylphenyl group, an ethoxyphenyl group, a propyloxyphenyl group, chlorophenyl group, or a chloromethylphenyl group.

The terms “group,” “radical,” and “substituent” may be used interchangeably. For example, the term “aryl,” “aryl group,” or “aryl substituent” may be used interchangeably. For purposes of this disclosure, an aryl group means a six carbon aromatic ring and the substituted variants thereof, including but not limited to, phenyl, 2-methyl-phenyl, xylyl, and 4-bromo-xylyl. Likewise a heteroaryl group means an aryl group where a ring carbon atom (or two or three ring carbon atoms) has been replaced with a heteroatom, preferably N, O, or S. As used herein, the term “aromatic” also refers to pseudoaromatic heterocycles which are heterocyclic substituents that have similar properties and structures (nearly planar) to aromatic heterocyclic ligands, but are not by definition aromatic; likewise, the term aromatic also refers to substituted aromatics.

The terms “alkyl group,” “alkyl substituent,” and “alkyl radical” may be used interchangeably. For purposes of this disclosure, an alkyl group is defined as a saturated hydrocarbon group, such as a C₁-C₁₄ group, that may be linear, branched, or cyclic, such as non-aromatic cyclic. Examples of such groups include, but are not limited to, methyl, ethyl, n-propyl, isopropyl, n-butyl, isobutyl, sec-butyl, tert-butyl, pentyl, iso-amyl, hexyl, octyl cyclopropyl, cyclobutyl, cyclopentyl, cyclohexyl, cyclooctyl, and the like including their substituted analogues. Substituted alkyl groups are groups in which at least one hydrogen atom of the alkyl group has been substituted with at least one functional group such as NR₂, OR, SeR, TeR, PR₂, AsR₂, SbR₂, SR, BR₂, SiR₃, GeR₃, SnR₃, PbR₃, and the like, or where at least one heteroatom has been inserted within an alkyl ring.

The term “alkenyl group” means a straight-chain, branched-chain, or cyclic hydrocarbon radical having one or more double bonds. These alkenyl groups may be optionally substituted. Examples of suitable alkenyl groups include, but are not limited to, ethenyl, propenyl, allyl, 1,4-butadienyl cyclopropenyl, cyclobutenyl, cyclopentenyl, cyclohexenyl, cyclooctenyl, and the like, including their substituted analogues.

The term “alkoxy group” or “alkoxide group” means an alkyl ether radical wherein the term alkyl is as defined above. Examples of suitable alkoxy groups include, but are not limited to, methoxy, ethoxy, n-propoxy, iso-propoxy, n-butoxy, iso-butoxy, sec-butoxy, tert-butoxy, phenoxy, and the like.

Where isomers of a named alkyl, alkenyl, alkoxide, or aryl group exist (e.g., n-butyl, iso-butyl, sec-butyl, and tert-butyl) reference to one member of the group (e.g., n-butyl) shall expressly disclose the remaining isomers (e.g., iso-butyl, sec-butyl, and tert-butyl) in the family. Likewise, reference to an alkyl, alkenyl, alkoxide, or aryl group without specifying a particular isomer (e.g., butyl) expressly discloses all isomers (e.g., n-butyl, iso-butyl, sec-butyl, and tert-butyl).

The following are examples of compounds having a structure represented by Formula (I): acetophenone, o-methylacetophenone, m-methylacetophenone, p-methylacetophenone, o-ethylacetophenone, m-ethylacetophenone, p-ethylacetophenone, o-chloroacetophenone, m-chloroacetophenone, p-chloroacetophenone, o-fluororoacetophenone, m-fluoroacetophenone, p-fluoroacetophenone, o-methoxyacetophenone, m-methoxyacetophenone, p-methoxyacetophenone, o-ethoxyacetophenone, m-ethoxyacetophenone, p-ethoxyacetophenone, 2,3-dimethylacetophenone, 2,4-dimethylacetophenone, 2,3,4-trimethylacetophenone, 2,3-diethylacetophenone, 2,4-diethylacetophenone, 2,3,4-triethylacetophenone, 2-chloro,3-methylacetophenone, 2-chloro, 4-methylacetophenone, 2,3-dichlorocetophenone, 2,4-dichloroacetophenone, 2,3,4-trichloroacetophenone.

Alkenylphenone

One or more embodiments of the present disclosure relate to methods of preparing and compositions including an alkenylphenone having a structure represented by Formula (IV):

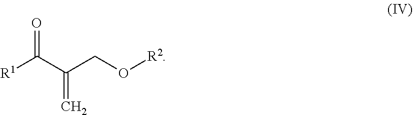

In some embodiments, R¹ may be a substituted or unsubstituted aryl group having 6 to about 20 carbons as described above. In one or more embodiments, R² may be a substituted or unsubstituted alkyl group. The alkenylphenone of the compositions may be prepared by a method including providing an arylacetone having the structure represented by Formula (I) and reacting the arylacetone with formaldehyde in the presence of a strong base catalyst to form an alkenylphenone composition.

In some embodiments, R² may be branched or unbranched. In some embodiments, R² may be a substituted or unsubstituted C₁-C₆ alkyl group. For example, R² may be a methyl group, an ethyl group, a propyl group, a butyl group, a pentyl group, or an hexyl group.

The following are examples of compounds having a structure represented by Formula (IV): phenyl 1-(methoxymethyl)vinyl ketone, o-methylphenyl 1-(methoxymethyl)vinyl ketone, m-methylphenyl 1-(methoxymethyl)vinyl ketone, p-methylphenyl 1-(methoxymethyl)vinyl ketone, o-ethylphenyl 1-(methoxymethyl)vinyl ketone, m-ethylphenyl 1-(methoxymethyl)vinyl ketone, p-ethylphenyl 1-(methoxymethyl)vinyl ketone, o-chlorophenyl 1-(methoxymethyl)vinyl ketone, m-chlorophenyl 1-(methoxymethyl)vinyl ketone, p-chlorophenyl 1-(methoxymethyl)vinyl ketone, o-methoxyphenyl 1-(methoxymethyl)vinyl ketone, m-methoxyphenyl 1-(methoxymethyl)vinyl ketone, p-methoxyphenyl 1-(methoxymethyl)vinyl ketone, o-ethoxyphenyl 1-(methoxymethyl)vinyl ketone, m-ethoxyphenyl 1-(methoxymethyl)vinyl ketone, p-ethoxyphenyl 1-(methoxymethyl)vinyl ketone, 2,3-dimethylphenyl 1-(methoxymethyl)vinyl ketone, 2,4-dimethylphenyl 1-(methoxymethyl)vinyl ketone, phenyl 1-(ethoxymethyl)vinyl ketone, o-methylphenyl 1-(ethoxymethyl)vinyl ketone, m-methylphenyl 1-(ethoxymethyl)vinyl ketone, p-methylphenyl 1-(ethoxymethyl)vinyl ketone, o-ethylphenyl 1-(ethoxymethyl)vinyl ketone, m-ethylphenyl 1-(ethoxymethyl)vinyl ketone, p-ethylphenyl 1-(ethoxymethyl)vinyl ketone, o-chlorophenyl 1-(ethoxymethyl)vinyl ketone, m-chlorophenyl 1-(ethoxymethyl)vinyl ketone, p-chlorophenyl 1-(ethoxymethyl)vinyl ketone, o-methoxyphenyl 1-(ethoxymethyl)vinyl ketone, m-methoxyphenyl 1-(ethoxymethyl)vinyl ketone, p-methoxyphenyl 1-(ethoxymethyl)vinyl ketone, o-ethoxyphenyl 1-(ethoxymethyl)vinyl ketone, m-ethoxyphenyl 1-(ethoxymethyl)vinyl ketone, p-ethoxyphenyl 1-(ethoxymethyl)vinyl ketone, 2,3-dimethylphenyl 1-(ethoxymethyl)vinyl ketone, 2,4-dimethylphenyl 1-(ethoxymethyl)vinyl ketone.

### Intermediates

One or more embodiments of the present disclosure relate to methods for preparing an alkenylphenone corrosion inhibiting composition, wherein the methods include forming intermediates having structures represented by Formulas (II) and (III):

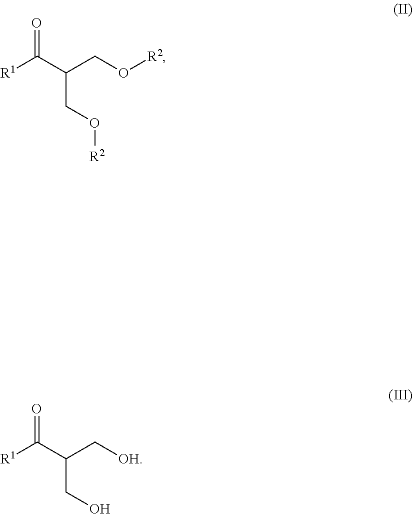

In one or more embodiments of the present disclosure, R¹ may be a substituted or unsubstituted aryl group having 6 to about 20 carbons as described above, and R² may be a substituted or unsubstituted alkyl group as described above.

These intermediates having structures represented by Formulas (II) and (III) result from the condensation of 2 formaldehyde molecule with 1 arylacetone molecule. Therefore, in some embodiments of the present disclosure, the molar ratio of arylacetone to formaldehyde may be 1:2 in order to result in the formation of an alkenylphenone having a structure represented by Formula (IV) via the formation of intermediates having structures of Formulas (II) and (III).

Condensation Reaction

In some embodiments, the methods of preparing an alkenylphenone having a structure represented by Formula (IV) include reacting an arylacetone having a structure represented by Formula (I) with formaldehyde in the presence of a strong base catalyst. In particular, in one or more embodiments, condensation of arylacetone with paraformaldehyde may be carried our using a strong base catalyst.

Examples of strong base catalysts which may be used are inorganic bases such as alkali metal hydroxides (e.g. sodium hydroxide), alkaline earth metal hydroxides (e.g. calcium hydroxide and magnesium hydroxide), alkali metal amides, alkali metal hydrides, and other organic strong bases, such as alkoxides and organolithiums.

For example, strong base catalysts may include, but are not limited to, sodium hydroxide (NaOH), potassium hydroxide (KOH), lithium hydroxide (LiOH), strontium hydroxide (Sr(OH)₂), barium hydroxide (Ba(OH)₂), rubidium hydroxide (RbOH), cesium hydroxide (CsOH), calcium hydroxide (Ca(OH)₂), and mixtures thereof. In some embodiments, the strong base catalyst may be selected from superbases including sodium ethoxide, butyl lithium, lithium diisopropylamide, lithium diethylamide, sodium amide, sodium hydride, lithium bis(trimethylsilyl)amide, and mixtures thereof.

In one or more embodiments of the present disclosure, the condensation of arylacetone with paraformaldehyde is carried out using 1 equivalent of the arylacetone having a structure represented by Formula (I) and 2 equivalents of paraformaldehyde. Accordingly, in the methods of preparing an alkenylphenone having a structure represented by Formula (IV), the arylacetone having a structure represented by Formula (I) is reacted with formaldehyde, these reactants being present in a molar ratio of 1:2, in the presence of a strong base catalyst.

In one or more embodiments of the present disclosure, the condensation of arylacetone with paraformaldehyde may be carried out using an alkyl alcohol compound R²OH, wherein R² may be a substituted or unsubstituted alkyl group. In particular, the condensation of arylacetone, formaldehyde, and alkyl alcohol is carried out to yield an alkenylphenone via the formation of aryl bis(hydroxyalkyl) ketone and aryl bis(alkoxyalkyl) ketone intermediates in the presence of a strong base catalyst. In some embodiments, R² may be branched or unbranched. In some embodiments, R²OH may be a substituted or unsubstituted C₁-C₆ alcohol compound. For example, R²OH may be a methanol group, ethanol, n-propanol, isopropanol, n-butanol, isobutanol, tert-butanol, pentanol, hexanol, or mixtures thereof.

Accordingly, the condensation of arylacetone with paraformaldehyde and alkyl alcohol in the presence of a strong base to provide the corresponding alkenylphenone may be represented by Scheme 1:

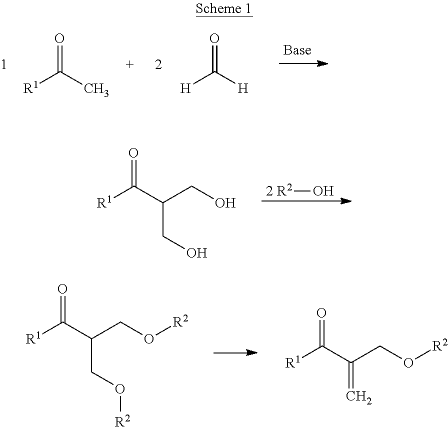

The condensation reaction may be carried out at suitable reaction temperatures ranging from about 0° C. to about 40° C., or from about 5° C. to about 35° C., or from about 10° C. to about 30° C., or from about 15° C. to about 25° C. These temperature conditions allow the scale-up of the condensation reaction at relatively mild conditions. The condensation reaction may be carried out for suitable durations ranging from about 10 hours to about 500 hours, or from about 15 hours to about 250 hours, or from about 25 hours to about 200 hours.

Corrosion Inhibition

In one or more embodiments of the present disclosure, the methods and compositions provide corrosion inhibitors, which may significantly reduce, mitigate, or inhibit the corrosion of the steel surface of oilfield equipment components, when the steel surface is in contact with compositions including an alkenylphenone having a structure represented by Formula (IV). In particular, the corrosion inhibitors according to some embodiments are suitable to reduce, mitigate, or inhibit iron sulfide deposition in formations and downhole tubing in carbonate sour gas and oil wells through the effective controlling of acid corrosion during acidizing treatments. Further, the compositions and methods result in corrosion inhibitors that may effectively mitigate the corrosion and significantly reduce the release of iron due to severe corrosion.

The compositions according to one or more embodiments may be used as corrosion inhibitors in acid stimulation fluids to protect the tubulars and prevent iron based scale precipitation. The corrosion inhibiting effect of an inhibitor can be tested in various ways. One direct method of testing is to use a test piece which is a sample of the steel to be protected, customarily referred to as a “coupon.” This coupon is exposed for a measured length of time to an acidic solution containing a known concentration of corrosion inhibitor. The loss in weight of the coupon is measured and expressed as weight loss per unit surface area. The coupon is also examined for localized pitting and the extent of pitting may be expressed as a numerical value: the so-called pitting index.

There are a number of other ways to measure corrosion by an acidic solution. These include linear polarization resistance measurement which was first proposed by M Stern and A L Geary in “Electrochemical Polarization: I. A Theoretical Analysis of the Shape of Polarization Curves” in J. Electrochem. Soc. Vol 104 pp 56-63 (1957) and followed by Stern: “A Method For Determining Corrosion Rates From Linear Polarization Data” in Corrosion, Vol. 14, No. 9, 1958, pages 440t-444t. In such tests a piece of the steel is used as an electrode and this electrode may be kept moving as a rotating disc, cylinder or cage to simulate flow of the corrosive solution over the steel.

When steel is exposed to a flow of an acidic composition, it is normal practice to test coupons of the steel with various concentrations of corrosion inhibitor in samples of the acidic composition. A concentration of inhibitor which produces an acceptably low weight loss and pitting index is identified and this concentration of inhibitor is then maintained in the flow of acidic composition to which the steel is exposed.

In one or more embodiments of the present disclosure, the methods and compositions provide corrosion inhibitors, which may significantly reduce the corrosion rate when high concentration of hydrochloric acid (HCl) is used for acid stimulation at high temperature. In particular, the corrosion inhibitors according to some embodiments are suitable to mitigate iron sulfide deposition in formations and downhole tubing in carbonate sour gas and oil wells via the effective controlling of acid corrosion during acidizing treatments.

Accordingly, the compositions according to some embodiments may be used in acid stimulation fluids to protect the tubulars and prevent iron based scale precipitation. As such, in one or more embodiments, are provided methods of inhibiting corrosion of a steel surface of an oilfield equipment component. In some embodiments, the methods may include adding a corrosion inhibitor including an alkenylphenone having a structure represented by Formula (IV) to acidizing fluids pumped downhole. In some embodiments, the corrosion inhibitor may be added simultaneously to acidizing fluids used downhole. In some embodiments, the corrosion inhibitor may be added apart from, such as sequentially, before or after, acidizing fluids used downhole. These methods may include contacting the steel surface with an aqueous solution comprising the composition including an alkenylphenone having a structure represented by Formula (IV). More particularly, in these methods, the steel surface may be contacted with an aqueous solution comprising HCl. In the aqueous solution, HCl may be at concentrations of from about 5%, or from about 10%, or from about 15% to about 50%, or to about 40%, or to about 35%, or to about 30%, or to about 28%. Further, the alkenylphenone having the structure of Formula (IV) may be present in the aqueous solution at a concentration of at least about 200 ppm, or at least about 25 ppm, or at least about 300 ppm, or at least about 350 ppm, or at least about 400 ppm, or at least about 450 ppm, or at least about 500 ppm. In addition, the steel surface may be contacted with the aqueous solution at a temperature of at least about 90° C. for at least about 4 hours. In the methods of inhibiting corrosion of a steel surface of an oilfield equipment component according to one or more embodiments, corrosion of the steel surface is inhibited by a corrosion inhibitor comprising the compositions containing an alkenylphenone having the structure of Formula (IV) at a corrosion inhibition efficiency (IE %) of at least 90%, or at least 91%, or at least 92%, or at least 93% in HCl, wherein the inhibition efficiency is expressed by IE %=(W₀−W)/W₀*100, wherein W₀ is a weight loss without the corrosion inhibitor and W is a weight loss in presence of the corrosion inhibitor.

### EXAMPLES

The following examples are merely illustrative and should not be interpreted as limiting the scope of the present disclosure.

### Examples 1-3 and Comparative Examples 1-7

Shown in Table 1 are the experimental details and conditions of base-catalyzed condensation reactions of acetophenone (Reactant 1a) or p-methoxyactophenone (Reactant 1b) with paraformaldehyde (Reactant 2). These reactants were provided in ratios of 1:2 and were mixed in the presence of a weak base K₂CO₃ (A) (Comparative Examples 1-7) or a strong base NaOH (B) (Examples 1-3) in methanol at various temperatures and during various reaction times to provide compositions containing phenyl 1-(methoxymethyl)vinyl ketone (Examples 1-2 and Comparative Examples 1-4) or p-methoxyphenyl 1-(methoxymethyl)vinyl ketone (Example 3 and Comparative Examples 5-7) as reaction products according to reaction Scheme 2:

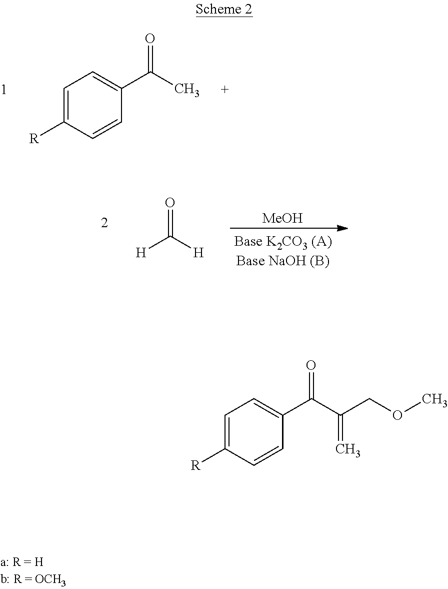

TABLE 1

| | | | Base | | | | |
|---|---|---|---|---|---|---|---|
| | Reactant | Reactant | A: K₂CO₃ | | | | Unreacted |
| | 1 | 2 | B: NaOH | MeOH | Temp | Time | 1 |
| | (mmol) | (mmol) | (mmol) | (mL) | (° C.) | (h) | (%) |
| CE1 | a: 250 | 250 | A: 2.4 | 27 | 25 | 72 | 64 |
| CE2 | a: 250 | 500 | A: 2.6 | 50 | 25 | 192 | 27 |
| CE3 | a: 250 | 500 | A: 2.6 | 50 | 50 | 144 | 30 |
| CE4¹ | a: 250 | 500 | A: 3.0 | 50 | 95 | 6 | 17 |
| E1 | a: 250 | 500 | B: 5.4 | 50 | 25 | 96 | 27 |
| E2 | a: 250 | 500 | B: 10 | 50 | 25 | 96 | 6 |
| CE5 | b: 250 | 500 | A: 2.7 | 80 | 25 | 240 | 51 |
| CE6 | b: 250 | 500 | A: 2.5 | 80 | 25 | 192 | 65 |
| CE7¹ | b: 250 | 500 | A: 3.0 | 50 | 95 | 6 | 30 |
| E3 | b: 250 | 500 | B: 10 | 80 | 25 | 120 | 13 |

¹These reactions are performed in autoclaves to reach reaction temperatures of 95° C. (as the boiling point of methanol being 65° C.).

The reaction temperature of Comparative Examples 4 and 7 were high and would thus require harsh and difficult scale-up conditions. In contrast, the condensation reactions of Examples 1-3 were carried out using a strong base NaOH at 25° C. Examples 2 and 3 provided the best yields as these Examples result in the least amounts of unreacted acetophenone and p-methoxyactophenone, respectively. In addition, the use of strong base NaOH as catalyst at room temperature may easily be scaled up without any operational issues.

### Examples 4-9

Table 2 shows the inhibition efficiencies of corrosion inhibitors in aqueous solutions containing phenyl 1-(methoxymethyl)vinyl ketone (CI-1) (Examples 4-6) and p-methoxyphenyl 1-(methoxymethyl)vinyl ketone (CI-2) (Examples 7-9) at various HCl concentrations and corrosion inhibitor concentrations.

The inhibition efficiencies were calculated according to the following procedure. A coupon was used for each example. The weight of the coupon was measured and the coupon was placed in a glass cell. Separately, aqueous solutions (50 ml) were prepared containing 15% and 28% HCl with and without corrosion inhibitor (CI-1 or CI-2), wherein when the corrosion inhibitor was present, it was in solution at concentrations of 200 or 500 ppm. The aqueous solutions were then heated at 90° C. for 4 hour. The aqueous solutions were then transferred to the glass cells containing the coupons.

After 4 hours, each coupon was rinsed with distilled water and dried. The weight of the coupon was then measured. The corrosion inhibiting efficiency was then calculated according to the following equation:

IE%=(W₀−W)/W₀*100

where W₀ is the weight loss without corrosion inhibitor and W is the weight loss in presence of corrosion inhibitor.

TABLE 2

| | | Inhibition Efficiency |
|---|---|---|
| Example | Solution | (EI %) |
| 4 | 15% HCl + 200 ppm CI-1 | 33.3 |
| 5 | 15% HCl + 500 ppm CI-1 | 97.1 |
| 6 | 28% HCl + 500 ppm CI-1 | 97.9 |
| 7 | 15% HCl + 200 ppm CI-2 | 93.1 |
| 8 | 15% HCl + 500 ppm CI-2 | 93.5 |
| 9 | 28% HCl + 500 ppm CI-2 | 97.6 |

As shown in Table 2, CI-1 provided high corrosion inhibition for both 15% and 28% HCl solutions at 90° C., in particular when the corrosion inhibitor concentration was 500 ppm. CI-2 showed high corrosion inhibition even at a concentration of 200 ppm in both 15% and 28% HCl solutions.

While only a limited number of embodiments have been described, those skilled in the art, having benefit of this disclosure, will appreciate that other embodiments can be devised which do not depart from the scope of the disclosure.

Although the preceding description has been described here with reference to particular means, materials and embodiments, it is not intended to be limited to the particulars disclosed here; rather, it extends to all functionally equivalent structures, methods and uses, such as those within the scope of the appended claims.

The presently disclosed methods and compositions may suitably comprise, consist or consist essentially of the elements disclosed and may be practiced in the absence of an element not disclosed. For example, those skilled in the art can recognize that certain steps can be combined into a single step.

Unless defined otherwise, all technical and scientific terms used have the same meaning as commonly understood by one of ordinary skill in the art to which these systems, apparatuses, methods, processes and compositions belong.

The ranges of this disclosure may be expressed in the disclosure as from about one particular value, to about another particular value, or both. When such a range is expressed, it is to be understood that another embodiment is from the one particular value, to the other particular value, or both, along with all combinations within this range.

The singular forms “a,” “an,” and “the” include plural referents, unless the context clearly dictates otherwise.

As used here and in the appended claims, the words “comprise,” “has,” and “include” and all grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

1. A method for preparing an alkenylphenone corrosion inhibiting composition comprising:
a. providing an arylacetone having a structure represented by Formula (I): 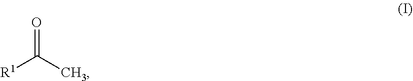
wherein R¹ is a substituted or unsubstituted aryl group having 6 to about 20 carbons; and
b. reacting the arylacetone with formaldehyde in the presence of a strong base catalyst to form an alkenylphenone composition.

2. The method of claim 1, wherein R¹ is a substituted aryl group, wherein the aryl group is substituted by one or more of an alkyl group, an alkenyl group, an alkoxide group, a halogen group, an halogenoalkyl, and an aryl group.

3. The method of claim 1, wherein R¹ is a phenyl group, a methoxyphenyl group, a tolyl group, a xylyl group, an ethylphenyl group, an isopropylphenyl group, an ethoxyphenyl group, a propyloxyphenyl group, chlorophenyl group, or a chloromethylphenyl group.

4. The method of claim 1, wherein reacting the arylacetone with formaldehyde is performed in the presence of an alkyl alcohol.

5. The method of claim 1, wherein reacting the arylacetone with formaldehyde is performed in the presence of methanol.

6. The method of claim 1, wherein 1 equivalent of the arylacetone is reacted with 2 equivalents of formaldehyde.

7. The method of claim 1, wherein the strong base catalyst comprises sodium hydroxide.

8. The method of claim 1, wherein the method further comprises forming an intermediate having a structure represented by Formula (II): 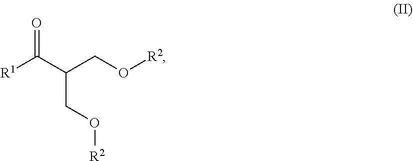
wherein R² is hydrogen or a substituted or unsubstituted alkyl group.

9. The method of claim 1, wherein the method further comprises forming an intermediate having a structure represented by Formula (III):

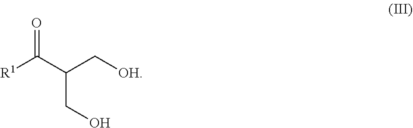

10. A composition comprising an alkenylphenone having a structure represented by Formula (IV): 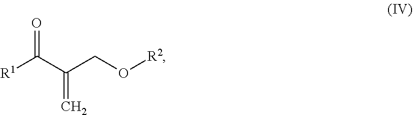
wherein R¹ is a substituted or unsubstituted aryl group having 6 to about 20 carbons, and R² is a substituted or unsubstituted alkyl group; the alkenylphenone being prepared by a method comprising:
a. providing an arylacetone having a structure represented by Formula (I) 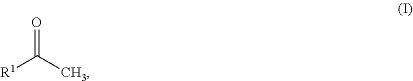
wherein R¹ is a substituted or unsubstituted aryl group having 6 to about 20 carbons; and
b. reacting the arylacetone with formaldehyde in the presence of a strong base catalyst to form an alkenylphenone composition.

11. The composition of claim 10, wherein R¹ is a substituted aryl group, wherein the aryl group is substituted by one or more of an alkyl group, an alkenyl group, an alkoxide group, a halogen group, an halogenoalkyl, and an aryl group.

12. The composition of claim 10, wherein R¹ is a phenyl group, a methoxyphenyl group, a tolyl group, a xylyl group, an ethylphenyl group, an isopropylphenyl group, an ethoxyphenyl group, a propyloxyphenyl group, chlorophenyl group, or a chloromethylphenyl group.

13. The composition of claim 10, wherein R² is a methyl group, an ethyl group, a propyl group, a butyl group, a pentyl group, or an hexyl group.

14. The composition of claim 10, wherein the alkenylphenone is prepared by reacting 1 equivalent of the arylacetone with 2 equivalents of formaldehyde.

15. The composition of claim 10, wherein the alkenylphenone is prepared in the presence of an alkyl alcohol R²—OH.

16. A method of inhibiting corrosion of a steel surface of an oilfield equipment component, the method comprising contacting the steel surface with an aqueous solution comprising a corrosion inhibitor comprising the composition of claim 10.

17. The method of claim 16, wherein the aqueous solution comprises hydrochloric acid (HCl).

18. The method of claim 16, wherein the alkenylphenone having the structure (IV) is present in the aqueous solution at a concentration of at least about 200 ppm.

19. The method of claim 16, wherein the steel surface is contacted with the aqueous solution at a temperature of at least about 90° C.

20. The method of claim 16, wherein corrosion of the steel surface is inhibited by the corrosion inhibitor at a corrosion inhibition efficiency (IE %) of at least 90% in HCl, wherein the inhibition efficiency is expressed by IE %=(W₀−W)/W₀*100, wherein W₀ is a weight loss without the corrosion inhibitor and W is a weight loss in presence of the corrosion inhibitor.
